(19) United States
(12) Patent Application Publication
(10) Pub. No.: US 2016/0348163 A1
(43) Pub. Date: Dec. 1, 2016

(54) APPARATUS FOR ANALYSING A MOLECULE

(71) Applicant: BASE4 INNOVATION LTD, Cambridge Cambridgeshire (GB)

(72) Inventors: Gareth PODD, Cambridgeshire (GB); Jekaterina KULESHOVA, Cambridgeshire (GB); Bruno Flavio Nogueira de Sousa SOARES, Cambridgeshire (GB)

(73) Assignee: BASE4 INNOVATION LTD, Cambridge, Cambridgeshire (GB)

(21) Appl. No.: 15/114,898

(22) PCT Filed: Jan. 30, 2015
(86) PCT No.: PCT/GB2015/050241

§ 371 Date: Jul. 28, 2016

(30) Foreign Application Priority Data
Jan. 31, 2014 (GB) 1401664.6
Sep. 12, 2014 (GB) 1416172.3

(51) Int. Cl. C12Q 1/68; G01N 21/65; G01N 27/447; G01N 33/487

(52) U.S. Cl. CPC C12Q 1/6869; G01N 33/48721; G01N 21/658; G01N 27/44791

(57) ABSTRACT

An apparatus for analysing a molecule comprising:•a substrate;•at least one nanopore provided in the substrate;•first and second reservoirs separated by the substrate for respectively providing and receiving the molecule;•a controller to induce the molecule to move by from the first reservoir to the second reservoir via the nanopore;•at least one nanostructure arranged in the nanopore and/or around the nanopore on one side of the substrate for producing a localised electromagnetic field by plasmon resonance;•at least one coating provided in the substrate and/or on one side of the substrate for cooling the substrate and/or reflecting electromagnetic radiation incident thereon;;•a source of electromagnetic radiation arranged on the side of the substrate bearing the nanostructure(s) to induce plasmon resonance in each nanostructure and•a detector to detect signals produced by interaction of the electromagnetic field with the molecule as it passes through the nanopore. The apparatus is especially suitable for analysing biomolecules such as nucleic acids or proteins by Raman spectroscopy.

The present invention relates to an improved plasmonic apparatus for investigating molecules. In one embodiment the apparatus is useful for determining the sequence of the constituent parts of biomolecules such as nucleic acids and proteins using Raman spectroscopy.

Next generation sequencing of genetic material is already making a significant impact on the biological sciences in general and medicine in particular as the unit cost of sequencing falls in line with the coming to market of faster and faster sequencing machines. For example, our co-pending application WO 2009/030953 discloses a new fast sequencer in which inter alia the sequence of nucleotide bases in a single- or double-stranded nucleic acid sample (e.g. naturally occurring RNA or DNA) is directly read by translocating the same through a nano-perforated substrate provided with plasmonic nanostructures juxtaposed within or adjacent the outlet of the nanopores. In this device, the plasmonic structures define detection windows comprising an electromagnetic field within which each nucleotide base (optionally labelled) is in turn induced to fluoresce or Raman-scatter photons in characteristic way by interaction with incident light. The photons so generated are then detected remotely, and converted into a data-stream whose information content is characteristic of the nucleotide base sequence itself. This sequence can then be recovered from the data-stream using computational algorithms embodied in corresponding software programmed into a microprocessor integral therewith or in a separate computing device attached thereto.

U.S. 2005/084912 discloses another apparatus for examining inter alia the sequence of nucleotide bases in a DNA sample as it translocates through a nanopore. Here, the apparatus suitably comprises a microscope detector consisting of an optical system comprising a nanolens assembly provided with one or more plasmon resonant particles. The optical system is arranged to be moveable and separate from the substrate bearing the nanopore.

U.S.2003/0036204 discloses a surface plasmon illumination system for producing bright nanometric light sources from apertures that are smaller than the wavelength of emitted light. At least some of the embodiments described include the presence of a metal layer on one side of the substrate. However the invention here is concerned with solving a somewhat different problem from that which we have encountered and discuss below.

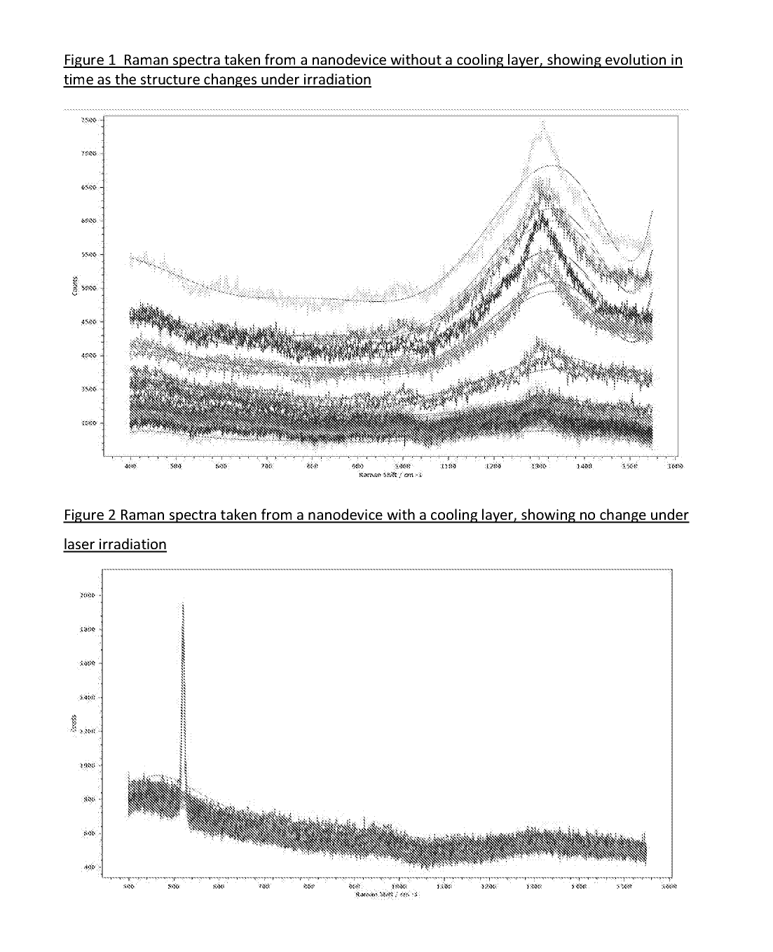

U.S. 2008/0239307 exemplifies a surface-enhanced Raman-scattering (SERS) method and a corresponding apparatus for sequencing polymeric biomolecules such as DNA, RNA or proteins. In the method disclosed, metallic nanostructures are caused to undergo plasmonic resonance thereby creating an electrical field enhancement near their surface which in turn can improve the Raman-scattering cross-section of any biomolecule located nearby. In FIG. 1 of this application an arrangement is shown in which the nanostructures are arranged on the surface of a nanoperforated substrate so that the biomolecule may be caused to translocate both through the nanopore and between the nanostructures.

WO 2011/076951 discloses a similar system for manipulating and analysing biomolecules using nanostructures and associated forces created by plasmon resonance. In one embodiment, it is taught that the system can also comprise a SERS detector.

U.S. Pat. No. 7,318,907 describes a nanoporous substrate having a metal layer on one side which is an attempt to solve a well-known problem in microscopy; the signal to noise problems associated with the backscattering of unfocused light. In this design the electromagnetic radiation is incident on the side of the substrate opposite to that on which the object is being detected.

U.S. 2007/014090 discloses a method for making a SERS biochip which involves depositing a conductive layer on a substrate followed by a noble metal layer and then a layer of alumina. Thereafter nano-wells are created by etching through the alumina and noble metal layers. However there appears to be no intention to create perforations in the conductive and substrate layers so that a molecule can translocate from one side of the chip to the other. Rather the intention appears to be to encapsulate the noble metal as far as possible so that so that non-contaminated nano-structures can be obtained.

Finally, JP 2010/0243267 and WO 2012/043028, WO 2013/179066, EP 2196796 and EP 2623960 disclose various devices in which biopolymers are caused to translocate through a perforated substrate coated with a thin metal film. Raman-scattering of light from the biopolymer is then analysed for example using a Raman detector.

One problem encountered with the apparatus described in WO 2009/030953 and indeed other nanopore plasmonic devices, especially when used to detect signals comprising Raman-scattered light, is that a high intensity light source such as a focused laser beam is required to excite the plasmonic nanostructures sufficiently enough to obtain a useful output signal. However, the nanostructures are efficient transducers of light into heat, and when illuminated with such sources can cause heating that can result in damage to the structures or analyte, and in some cases to the boiling of the solution in which the analyte is suspended (see for example; ACS Nano 4, 2 709-716 (2010) and Nano Letters 2013, 13, 1029-103). Furthermore, if the structures become too hot the risk of degrading the nanostructures and/or the substrate on which they are located becomes very significant. We have now overcome this problem by providing an efficient means to dissipate heat from the substrate on which the nanostructures are located. Thus, according to a first aspect of the invention there is provided an apparatus for analysing a molecule characterised by comprising:
a substrate;
at least one nanopore provided in the substrate;
first and second reservoirs separated by the substrate for respectively providing and receiving the molecule to be analysed;
a controller to induce the molecule to move by from the first reservoir to the second reservoir via the nanopore;
at least one nanostructure arranged in the nanopore and/or around the nanopore on one side of the substrate for producing a localised electromagnetic field by plasmon resonance;
at least one coating provided in the substrate and/or on one side of the substrate for cooling the substrate or reflecting electromagnetic radiation incident thereon;
a source of electromagnetic radiation arranged on the side of the substrate bearing the nanostructure(s) to induce plasmon resonance in each nanostructure and
a detector to detect signals produced by interaction of the electromagnetic field with the molecule as it passes through the nanopore.

The apparatus of the present invention can be used for investigating any molecule which is able to emit a characteristic signal when caused to interact with an electromagnetic field produced by plasmon resonance. Such molecules can include for example organic polymers, toxic residues (e.g. pesticides and herbicides), explosive residues, noxious gases and the like. In one embodiment, the apparatus is designed to be used to investigate biomolecules, for example biopolymers. For example, the apparatus can be used to determine the sequence of nucleotides in nucleic acids or the sequence of amino acids in proteins by detecting signals characteristic of the nucleotide bases in the nucleic acid or the amino acids. In another embodiment, the characteristic signal being produced by the molecule or the constituent parts referred to above is a Raman-scattering signal characteristic of the vibrations of particular bonds therein. Thus in a preferred, second aspect of the invention there is provided an apparatus for analysing a biopolymer selected from nucleic acids and proteins using Raman spectroscopy characterised by comprising:
a substrate;
at least one nanopore provided in the substrate;
first and second reservoirs separated by the substrate for respectively providing and receiving the biopolymer;
a controller to induce the biopolymer to move by from the first reservoir to the second reservoir via the nanopore;
at least one nanostructure arranged in the nanopore and/or around the nanopore on one side of the substrate for producing a localised electromagnetic field by plasmon resonance;
at least one coating provided in the substrate and/or on one side of the substrate for cooling the substrate or reflecting electromagnetic radiation incident thereon;
a source of electromagnetic radiation arranged on the side of the substrate bearing the nanostructure(s) to induce plasmon resonance in each nanostructure and
a detector to detect Raman-scattering signals produced by interaction of the electromagnetic field with the constituent parts of the biopolymer as the biopolymer passes through the nanopore.

In embodiments of the first and second aspects of the invention, each apparatus may further include a computing means for processing a data-stream from the detector and/or microfluidic pathways to deliver the molecule to and away from the first and/or second reservoirs.

The substrate employed in the apparatus is typically comprised of a dielectric material such as glass, silicon, silicon nitride, non-conducting polymer or the like. It may for example be in the form of one or more relatively thin membranes. In one embodiment, to add mechanical rigidity, the substrate may alternatively comprise a composite of one or more dielectric materials and one or more resilient materials. In this case, the dielectric material should comprise at least one of the outermost surfaces of the substrate; in particular the surface of the substrate adjacent the nanostructure(s).

The substrate may comprise a layered composite with each layer having a different refractive index relative to those juxtaposed either side of it. In one embodiment the refractive index of each layer reduces progressively in a direction away from the surface of the substrate adjacent the nanostructure(s). In another the various layers are comprised of different silicon materials for example elemental silicon, silicon dioxide, silicon nitride etc. In yet another, the layers are of different thicknesses.

In one embodiment, the substrate comprises a sheet of dielectric material or a composite thereof which is nanoperforated with nanopores. Preferably, the substrate may take the form of a nanoperforated membrane bonded to a perforated support (which may be a dielectric) to add mechanical rigidity. In another embodiment these nanopores are arranged so that they comprise an array; for example a regular array. The exact number of nanopores employed is not critical although it is clearly desirable that their number and density is such that it does not adversely compromise the mechanical integrity of the substrate. Suitably, the apparatus is one which comprises a plurality of nanopores.

Typically, each nanopore is generally tubular, for example cylindrical, in shape with an average internal diameter of between 2 nm and 100 nm, preferably 2 nm to 50 nm, 2 nm to 30 nm, 2 nm to 20 nm or 2 nm to 10 nm. The nanopores however may have a variable diameter as for example the case where they are tapered from the inlet towards the outlet or where the pore contains a larger chamber which can function as the degradation zone. Furthermore, the internal surfaces of the nanopores may also be chemically modified; for example to make them relatively more or relatively less hydrophilic or to attach moieties which are able to bind reversibly to the translocating molecule either chemically or, in the case of a nucleic acid sample such as DNA or RNA, by hybridisation.

The apparatus further comprises first and second chambers which respectively provide and receive a sample of the molecule to be analysed. In one practical embodiment, the arrangement of first and second chamber and intervening substrate is fabricated by arranging a nanoperforated sheet of the substrate within a larger chamber so as to divide it into two. Typically, each first and second chamber will have its own inlets and outlets so that a sample containing the molecule can be made to flow through the first chamber and the contents of the second chamber flushed. In one embodiment these inlets and outlets are integrated into corresponding microfluidic pathways.

The controller for causing the molecule to pass through the nanopore can for example work by electrical, mechanical, magnetic or osmotic effects. In one embodiment however, where the apparatus is adapted to analyse molecules, suitably biomolecules or biopolymers, bearing an anionic charge, the controller suitably comprises electrodes for causing electrophoretic transfer of the molecule. In one embodiment, this is achieved by providing a pair or pairs of electrodes of opposite polarity in the first chamber and second chamber or on opposite sides of the substrate and applying a potential difference therebetween. In one embodiment, the application of this potential difference can be time-dependent so that control of the molecule through the nanopore can be achieved. In another embodiment the polarity of the field created by the potential difference can be reversed to allow the analyte to pass back and forth through the nanopore.

Arranged in the nanopore or on one side of the substrate are nanostructures capable of generating a strong localised electromagnetic field when caused to undergo plasmon resonance.

In one embodiment, the nanostructures are preferably nanoparticulate in form and can in principle be of any shape including spherical polyhedral, prismatic or even amorphous. In one useful embodiment, pairs of nanostructure each generally wedge-shaped are arranged on the surface of the substrate about each nanopore with the most acute-angled apex of each being located closest to the nanopore. This ‘bow-tie’ configuration has the particular advantage of focusing the electromagnetic field around the nanopore. Alternatively, the nanostructure may be annular or substantially annular as in the shape of a donut with the centre of the donut located above a nanopore opening so that in effect the molecules are also caused to translocate though the nanostructure when the apparatus is in use. In this configuration, the internal diameter of the annulus is in general similar to that of the nanopore opening. In another embodiment the nanostructures may optionally be contained within a waveguide or associated with an antenna arrangement to increase the coupling of the excitation to the structure. An example of this arrangement comprises a bow-tie arrangement contained within an annular waveguide.

The nanostructures themselves are typically fabricated from metals or dielectric materials coated with metal. Metals which can be employed are those capable of undergoing plasmon resonance to a significant extent, for example, gold, silver, copper, aluminium, platinum, palladium, molybdenum and chromium and alloys thereof. Preferably, the metal used will be gold, silver, copper, aluminium or an alloy thereof. In one embodiment each nanostructure may have attached to its surface binding sites which are specifically adapted to capture and release the molecule or a constituent part thereof. Each nanostructure should be made to a size that makes it resonant with the source of electromagnetic radiation. Typically this will mean a maximum dimension of less than 1000 nm, preferably less than 500 nm and most preferably in the range 50 to 350 nm. Where the nanostructures are arranged in pairs, each may be spaced apart from its pair by up to 25 nm, preferably up to 10 nm, more preferably up to 5 nm.

Arranged on one side of the substrate or embedded therein is at least one coating for removing heat generated in or around the nanostructures and/or reflecting electromagnetic radiation incident on the substrate and/or coating in the manner of a mirror. In the case of the latter, this can enable the collection angle for the signal photons to be reduced and/or to modify the plasmonic modes of the nanostructures to cause further electromagnetic field enhancement. In one embodiment, this coating comprises a cooling means itself consisting of a layer of a material having a high thermal conductivity (e.g. a metal or silicon) arranged on a second side of the substrate opposite a first side bearing the nanostructures. In another, the coating comprises a layer of highly reflective material arranged on the second side. In yet another embodiment, the coating fulfils both these functions. For example, in one such configuration, the nanostructures are arranged around the outlets of the nanopores and the coating around the inlet side. In the case where the coating is a metal, it may act both as a cooling layer and as a mirror for reflecting electromagnetic radiation transmitted through the substrate back towards the detector, with the additional benefit of an enhanced optical signal. In this case, the metal may either be the same or different from the metallic material comprising the nanostructures. Here, it is preferred that the thickness of the substrate is less than 100 nm, more preferably less than 50 nm. In another embodiment, the coating may comprise one or more layers in a composite substrate. In yet another embodiment, the coating extends into the nanopore. In another, the nanostructures comprise dielectric structures covered with a layer of the metal capable of undergoing plasmon resonance and the coating is embedded in the dielectric core and extending therefrom into the bulk of the substrate. In another embodiment the coating in any of these configurations is attached to a larger heat-sink and/or a peltier cooler. In a final embodiment graphene is used instead of a conducting metal.

Alternatively, the coating comprises a series of microfluidic channels in the substrate through which a coolant is continuously passed.

The source of electromagnetic radiation for inducing plasmon resonance in each nanostructure is suitably substantially monochromatic, of high intensity and highly focused. A particularly suitable source of such radiation is a laser together with associated optics for focusing a laser beam. The electromagnetic radiation is characterised by the fact that in the apparatus it first impinges on the side of the substrate bearing the nanostructure(s) and, in the case where it is reflected by the coating, is substantially only so reflected after passing through the substrate. In other words as a matter of apparatus geometry, the electromagnetic radiation source and the substrate are arranged on the same side of the substrate.

The detector is one which is able to detect signals from the interaction of the molecule with the electromagnetic field generated by the nanostructures. Where Raman spectroscopy is the basis for detection the detector is suitably a photodetector capable of detecting characteristic Raman scattered light. For example, if the molecule is a nucleic acid, such as DNA, one possibility is that the detector is capable of detecting four different wavelengths each uniquely characteristic of a vibrational mode of the constituent adenine, thymine, guanine and cytosine nucleotide bases. The detection of additional wavelengths can also be used to allow the characterisation of modified nucleotide bases, for example methylated cytosine or adenine. In one embodiment, each molecule may also be detected at multiple wavelengths to ensure it is detected reliably. Typically the wavelengths at which this detection occurs have the value λ_{d} wherein:

λ_{d}=λₑ+λ_{b} (Stokes shift)

λ_{d}=λₑ−λ_{b} (Anti-Stokes shift)

λₑ is the wavelength of the incident light (typically occurring in the visible or near ultra-violet) and λ_{b} is the wavelength of the characteristic vibrational mode of the base (typically occurring in the infra-red). In one embodiment, detection is carried out on Stokes-shifted scattered light. Alternatively, it is possible to detect Anti-Stokes-shifted scattered light but in such an approach the molecule will need to be first pumped up to a low-lying excited vibrational state by a second source of electromagnetic radiation of the correct wavelength. In both cases, the signal from the molecule is enhanced by the presence of the nanoparticles by way of the Surface Enhanced Raman Effect (SERS). For the nucleotide bases characteristic of DNA, examples of the vibrational modes most commonly detected by Raman-scattering are those associated with the in-phase ring breathing vibration. These occur at frequency shifts of 485 to 505 cm⁻¹ for thymine, 675 to 690 cm⁻¹ for guanine, 775 to 800 cm⁻¹ for cytosine, and 724 to 732 cm⁻¹ for adenine.

The exact form of the Raman detector employed in the apparatus is not critical and can suitably comprise for example a Raman spectrometer, a photodetector, a single photon avalanche diode, an electron-multiplying charge-coupled device or a complementary metal oxide semiconductor device. Preferably the Raman detector is tuneable to a Raman-scattering frequency characteristic of at least one of the mononucleotides. The detector can additionally be attached to a computing means such as microprocessor or PC to process the signal derived therefrom. In the case where a plurality of nanopores is employed it is envisaged that the optics will be multiplexed to allow parallel detection from all the nanopores simultaneously. Alternatively, it is possible to scan the nanopores using a raster arrangement or using a grating in a spectrometer arrangement.

The apparatus of the present invention can be of either a unitary or modular design. One convenient modular design comprises (a) a chip comprising the substrate, the coating, first and second chambers and the plasmon resonator and (b) a housing which comprises the detector, e.g. the Raman detector, and optionally is adapted to receive the chip. In one embodiment, the housing comprises the source of electromagnetic radiation, the detector and any associated electrical circuitry. The housing may further include the microprocessor or a connector for attaching a computer. The chip, housing or both may further comprise the controller or elements thereof.

Thus in an embodiment of the invention there is provided a chip in accordance with the apparatus of the present invention comprising the substrate, the coating, first and second chambers, the plasmon resonator and optionally elements of the controller. Suitably the chip is at least in part made of plastic and designed to be disposable after a one-time use. It is envisaged that the chip and housing can be sold separately.

As mentioned above the apparatus of the present invention is especially suitable for sequencing (1) the constituent nucleotides of a nucleic acid or (2) the constituent amino acids in a protein. Thus in an embodiment of the invention there is provided a method for sequencing a nucleic acid or protein by Raman spectroscopy which comprises the steps of (1) translocating the nucleic acid or protein in an aqueous medium through a substrate comprising at least one nanopore and at least one plasmonic nanostructure arranged in the nanopore and/or around the nanopore on one side of the substrate and (2) detecting Raman-scattering signals produced by interaction of an electromagnetic field induced in each plasmonic nanostructure with the constituent parts of the nucleic acid or protein as each part passes through the nanopore characterised in that the method further comprises the step of removing heat generated in the nanostructures or reflecting electromagnetic radiation using a coating provided within the substrate and/or on one side of the substrate.

Examples of nucleic acids which can be sequenced by this method include naturally-occurring DNA or RNA or synthetic analogues thereof. The method is especially useful for sequencing long polynucleotide fragments derived from human or mammalian DNA or RNA.

The present invention is now illustrated with reference to the following comparative experiments.

Two chips suitable for use in a DNA sequencing apparatus of the type described above were prepared. They comprised a nanoperforated silicon nitride membrane on one side of which was deposited an array of gold bow-tie nanostructure pairs. Each nanostructure in the pair has a maximum dimension 150 nm. The silicon membrane employed was 30 nm thick.

The second chip prepared was identical to the first except that on the side of the membrane opposite the gold nanostructures there was deposited a layer of thermally conductive silicon 250 um thick.

To test the two chip's relative performance, the sides of the membrane on which the gold nanostructures were located were each subjected to 300, one-second scans with a Helium-Neon laser operating at a wavelength of 785 nm and an objective power of 96 mW. The results are shown respectively in FIGS. 1 and 2. In this case every tenth scan is displayed. For the first chip, growth in a background signal was observed over time which was found to correlate strongly with the tail-off in detection performance of an identical chip employed to identify the nucleotide bases in a DNA analyte by Raman spectroscopy. In the case of the second chip, despite the presence of a sharp peak characteristic of silicon at 520 cm⁻¹, the background signal remained relatively constant over the experiment as did the detection performance of an identical chip used for detection purposes.

1. An apparatus for analysing a molecule, the apparatus comprising:
a substrate;
at least one nanopore provided in the substrate;
first and second reservoirs separated by the substrate for respectively providing and receiving the molecule;
a controller to induce the molecule to move from the first reservoir to the second reservoir via the nanopore;
at least one nanostructure arranged in the nanopore and/or around the nanopore on one side of the substrate for producing a localised electromagnetic field by plasmon resonance;
at least one coating provided in the substrate and/or on one side of the substrate for cooling the substrate and/or reflecting electromagnetic radiation incident thereon;
a source of electromagnetic radiation arranged on the side of the substrate bearing the nanostructure(s) to induce plasmon resonance in each nanostructure and
a detector to detect signals produced by interaction of the electromagnetic field with the molecule as it passes through the nanopore.

2. The apparatus as claimed in claim 1,
wherein the molecule is a biopolymer selected from the group consisting of nucleic acids and proteins, and
wherein the signals are Raman-scattering signals produced by the interaction of the electromagnetic field with the constituent parts of the biopolymer as the biopolymer passes through the nanopore.

3. The apparatus as claimed in claim 1, wherein the coating comprises a layer of metal or graphene.

4. The apparatus as claimed in claim 1, wherein the coating and nanostructure(s) are located in or on opposite sides of the substrate.

5. The apparatus as claimed in claim 1, wherein the coating is embedded within the nanopores of the substrate.

6. The apparatus as claimed in claim 1, wherein the coating is attached to a heat sink or a peltier cooler.

7. The apparatus as claimed in claim 1, wherein the coating is located within nanostructures comprising a dielectric core covered with an outer layer of metal capable of undergoing plasmon resonance.

8. The apparatus as claimed in claim 1, wherein the controller comprises a pair or pairs of electrodes of opposite polarity arranged in the first and second reservoirs on either side of the substrate.

9. The apparatus as claimed in claim 1, wherein the nanostructures are arranged on the substrate in a bow-tie configuration.

10. The apparatus as claimed in claim 1, wherein the at least some of the nanostructures are contained within a waveguide or associated with an antenna system.

11. The apparatus as claimed in claim 1, wherein the detector is a detector for detecting Raman-scattered radiation.

12. The apparatus as claimed in claim 1, further comprising a computing means for analysing the output from the detector.

13. The apparatus as claimed in claim 1, further comprising a chip comprising the substrate, the first and second reservoirs and the source of electromagnetic radiation and a housing comprising the detector.

14. The apparatus as claimed in claim 13, wherein the chip, the housing or both further comprise the controller.

15. A chip comprising
a substrate;
at least one nanopore provided in the substrate;
first and second reservoirs separated by the substrate for respectively providing and receiving the nucleic acid or protein;
a controller to induce the nucleic acid or protein to move from the first reservoir to the second reservoir via the nanopore;
at least one nanostructure arranged in the nanopore and/or around the nanopore on one side of the substrate for producing a localised electromagnetic field by plasmon resonance;
at least one coating provided in the substrate and/or on one side of the substrate for cooling the substrate and/or reflecting electromagnetic radiation incident thereon; and
a source of electromagnetic radiation arranged on the side of the substrate bearing the nanostructure(s) to induce plasmon resonance in each nanostructure.

16. A method for sequencing a nucleic acid or protein, the method comprising
(1) introducing the nucleic acid or protein into the first reservoir of the apparatus according to claim 1,
(2) translocating the nucleic acid or protein in an aqueous medium through the substrate,
(3) detecting the signals produced by the interaction of the electromagnetic field with constituent parts of the nucleic acid or protein as each part passes through the nanopore, and
(4) removing heat generated in the nanostructures or reflecting the electromagnetic radiation using the coating.

17. A method for sequencing a nucleic acid or protein by Raman spectroscopy, the method comprising
(1) translocating the nucleic acid or protein in an aqueous medium through a substrate comprising at least one nanopore and at least one plasmonic nanostructure arranged in the nanopore and/or around the nanopore on one side of the substrate,
(2) detecting Raman-scattering signals produced by interaction of an electromagnetic field induced in each plasmonic nanostructure with the constituent parts of the nucleic acid or protein as each part passes through the nanopore, and
(3) removing heat generated in the nanostructures or reflecting electromagnetic radiation using a coating provided within the substrate and/or on one side of the substrate.

18. The apparatus as claimed in claim 13, wherein the controller comprises a pair or pairs of electrodes of opposite polarity arranged in the first and second reservoirs on either side of the substrate, and wherein the chip, the housing or both further comprise the controller or elements thereof.
